(19) United States
(12) Patent Application Publication
(10) Pub. No.: US 2003/0031671 A1
(43) Pub. Date: Feb. 13, 2003

(54) Methods of treating colon cancer utilizing tumor-specific antibodies

(76) Inventors: Sydney Welt, New York, NY (US); Lloyd J. Old, New York, NY (US)

Correspondence Address: FULBRIGHT & JAWORSKI, LLP, 666 FIFTH AVE, NEW YORK, NY, 10103-3198, US

(21) Appl. No.: 09/920,147

(22) Filed: Aug. 1, 2001

(51) Int. Cl. A61K 39/395

(52) U.S. Cl. 424/155.1; 424/178.1

(57) ABSTRACT

This invention relates to methods of reducing the effects of colon cancer tumors. Various agents are conjugated to PEG(polyethylene glycol)-conjugated antibody which are specific for colon cancer cells. The conjugates are administered to patients having colon cancer such that the effects of the cancer are reduced.

### FIELD OF THE INVENTION

This invention relates to a method of reducing the effects of colon cancers, utilizing at least one monoclonal antibody conjugated to PEG. Specifically, at least one PEG conjugated monoclonal antibody is utilized in conjunction with an anti-tumor drug, a peptide which inhibits DNA tumor activity or a radioisotope in the treatment of colorectal carcinoma. This invention further relates to a method of delivering genetic material to DNA of tumor cells and to a method of delivering anti-cancer agents to nuclei of colon tumor cells, as well as to PEG conjugated monoclonal antibodies which are specific for A33 antigen; an antigen found on colon cancer cells.

### BACKGROUND OF THE INVENTION

Colorectal carcinoma is a malignant neoplastic disease. There is a high incidence of colorectal carcinoma in the Western world, particularly in the United States. Tumors of this type often metastasize through lymphatic and vascular channels. Many patients with colorectal carcinoma eventually die from this disease. In fact, it is estimated that 62,000 persons in the United States alone die of colorectal carcinoma annually.

To date, systemic therapies and chemotherapies have been developed for the treatment of colorectal cancer. However, no therapies have exhibited sufficient anti-tumor activity to prolong the survival of colorectal carcinoma patients with metastatic disease with any degree of reliability. As a result, a need still exists to develop methods for the successful treatment of colorectal carcinoma. Recently, the A33 antibody has been shown to be a promising reagent in the treatment against colorectal carcinoma.

Monoclonal antibody A33 (mAb A33) recognizes a newly characterized cell-surface differentiation antigen of approximately 43 kDa molecular weight that belongs to the immunoglobulin superfamily. It is expressed on normal human gastrointestinal epithelium and on about 95% of primary or metastatic colon cancers, but is absent in most other normal tissues (Catimel, B. et al., J Biol Chem, 271: 25664-25670, 1996; Heath, J. K. et al., Proc Natl Acad Sci USA, 94: 469-474, 1997). Some colon cancer cell lines, such as SW1222, express large amounts of the A33 antigen, binding up to 800,000 antibody molecules per cell. Upon binding to the A33 antigen, the mAb A33 is internalized into a yet incompletely characterized vesicular compartment, and a significant fraction of the internalized antibody is recycled back to the cell surface (Daghighian, F. et al., J. Nucl. Med., 37:1052-1057, 1996). The A33 antigenic system has been the focus of several clinical studies in patients with colon cancer. Phase I/II clinical trials have shown that murine mAb A33 (i) localizes with high specificity to colon cancer (Welt, S. et al., J. Clin. Oncol, 8: 1894-1906, 1990); (ii) is retained for prolonged periods of up to 6 weeks in the cancer but clears within 5 to 6 days from normal colon (Welt, S. et al., J Clin Oncol, 12: 1561-1571, 1994); and (iii) has anti-tumor activity as a carrier of ¹²⁵I or ¹³¹I (Welt, S. et al., J Clin Oncol, 12: 1561-1571, 1994). A humanized version of the A33 antibody (hu A33) has been constructed (King, D. J. et al., Br. J. Cancer, 72: 1364-1372, 1995) and is currently being evaluated in clinical phase I trials (Welt, S. et al., Proc Annu Meet Am Soc Clin Oncol, 16: A15631997).

One problem associated with non-human (rodent) specific antibodies is rejection by the host immune system, which may treat such antibodies as foreign antigens. The result of this recognition is that such antibodies cannot be administered to the patients repeatedly because of the formation of antibodies against these agents. Repeated injections with A33 may lead to the formation of HAMA (human anti-mouse antibody) and a generalized immediate type-III hypersensitivity reaction to the antibody moiety of the conjugate. To overcome this problem, researchers have humanized the A33 antibody. Chimeric or humanized mouse-monoclonal antibodies (Rybak, S. M. et al., Proc Natl Acad Sci USA, 89: 3165-3169,1992; King, D. J. et al., Br. J. Cancer, 72: 1364-1372, 1995.) and antibodies derived from human DNA libraries by phage display (Hoogenboom, H. R. et al., Immunol. Rev., 130:41-68: 41-68, 1992) have been developed to overcome the host immune response. Furthermore, humanization may not be feasible with fusion proteins of antibodies and effector proteins such as enzymes or toxins. First, many of these effector proteins, such as bacterial toxins (e.g., Pseudomonas endotoxin, (Reiter, Y. and Pastan, I. Trends Biotechnol., 16: 513-520, 1998) Staphylococcus superantigen (Gidlof, C. et al., Eur. J. Haematol., 60: 233-239, 1998)) or prodrug-activating enzymes (e.g., cytosine deaminase (Senter, P. D. et al., Bioconjug. Chem., 2: 447-451, 1991) and beta-lactamiase (Kerr, D. E., et al., Cancer Res., 55: 3558-3563, 1995)), are of non-human origin and have no human counterpart. Second, even if the components of these constructs were fully human-derived or humanized, their junction region may still represent immunogenic epitopes. Thus, repeated injections of humanized antibodies may lead to the formation of HAHA (human anti-human antibody).

### SUMMARY OF THE INVENTION

This invention is directed to methods and reagents using PEG-conjugated antibodies. In this application a PEG-conjugated antibody is also referred to an a PEGylated antibody. In one embodiment, the invention is directed to a method of reducing the effects of colon cancer comprising the administration of tumor specific PEGylated antibodies conjugated to an anti-cancer agent. Specifically, the antibody is conjugated to at least one PEG molecule and at least one anticancer agent. Alternatively, at least one conjugate of a PEGylated antibody which is specific for colon cancer cells and peptides which inhibits DNA activity of said cells, such as peptides which inhibit activation pathwasys such as STAT pathways, tyrosine kinase pathways, and so forth is administered in a pharmaceutically effective amount so as to reduce the effects of colon cancer. Another method of reducing the effects of colon cancer comprises administering a pharmaceutically effective amount of at least one conjugate of a PEGylated antibody which is specific for said tumor and a radioisotope.

This invention is also directed to PEGylated antibodies, including monoclonal, humanized, chimeric, trimeric, heteromeric, single chain antibodies and antibody fragments, which can be utilized to reduce the effects of colon cancers.

Conjugation of therapeutic drugs with poly[ethylene glycol] (PEG)¹ has been successfully employed to increase their circulating half-life and solubility as well as to reduce immunogenicity and toxicity. This approach has been applied clinically, allowing bacterial enzymes such as L-asparaginase to be administered repeatedly (Abuchowski, A. et al., Cancer Biochem. Biophys., 7: 175-186, 1984; Ettinger, L. J. et al., Cancer, 75: 1176-1181, 1995). PEG-conjugation of enzymes and smaller proteins such as interleukin-2 has led to enhanced serum half-life, resulting in smaller doses and longer administration intervals without loss of clinical effectiveness (Teppler, H. et al., J. Exp. Med., 177: 483-492, 1993). ¹ Abbreviations: Ab, antibody; F(ab), F(ab′)₂, antibody fragments comprising one or two antigen-binding domains, respectively; CDR, complementarity-determining region; huA33, hu3S193, humanized A33 and 3S193 antibodies, respectively; PBS, phosphate buffered sodium chloride solution; PEG 5, PEG 12, and PEG 20, methoxy-polyethylene glycol succinimidyl-succinate of Mr 5000, Mr 12,000 and Mr 20,000, respectively.

While increased serum half-life and reduced immunogenicity are widely accepted as general effects of PEGylation on drug molecules, its role in drug targeting to tumors is less clear. Several groups have reported increased passive tumor uptake of a variety of molecules such as liposomes (Vaage, J. et al., Br. J. Cancer, 75: 482-486, 1997), a small-molecule drug (Westerman, P. et al., Int. J. Cancer, 76: 842-850, 1998), or an antibody without specificity for any tumor-associated antigen (Senter, P. D. et al., Bioconjug. Chem., 6: 389-394, 1995), and suggested a potential role for PEG in increasing passive targeting. This effect has been proposed to be due to the leakiness of tumor neovasculature (Jain, R. K. Cancer Res, 50: 814s-819s, 1990), facilitating extravasation into tumors, but not into normal tissue with intact vasculature. The tumor to blood ratios were markedly reduced compared to the non-PEGylated product, and in one study that investigated localization histologically, the drug was predominantly found in or around tumor vasculature (Westerman, P. et al., Int. J. Cancer, 76: 842-850, 1998).

In active targeting, i.e., tumor uptake due to specific binding, the effect of PEGylation on tumor localization appeared to depend on the protein size: tumor localization of PEGylated complete IgG was reduced compared to the native antibody (Kitamura, K. et al., Cancer Res, 51: 4310-4315, 1991), and several groups have reported increased absolute tumor uptake of Fab′ or Fab′₂ fragments (Kitamura, K. et al., Cancer Res, 51: 4310-4315, 1991; Delgado, C. et al., Br. J. Cancer, 73: 175-182,1996; Eno-Amooquaye, E. A. et al., Br. J. Cancer, 73: 1323-1327,1996). As in passive targeting, reduced tumor to blood ratios were observed after PEGylation of Fab′ or Fab′₂ fragments (Delgado, C. et al., Br. J. Cancer, 73: 175-182, 1996; Eno-Amooquaye, E. A. et al., Br. J. Cancer, 73: 1323-1327, 1996). Two factors are thought to be responsible for this effect, reduced clearance rate (Eno-Amooquaye, E. A. et al., Br. J. Cancer, 73: 1323-1327, 1996) and reduced diffusion of macromolecules. Diffusion characteristics have been investigated in detail for non-modified antibodies and F(ab)2 fragments, and several authors have concluded that tumor-directed macromolecules in general will not be able to achieve homogeneous distribution in tumor tissue due to elevated convective intratumoral pressure and low diffusion capacity of macromolecules (Jain, R. K. Cancer Res, 50: 814s-819s, 1990; Francis, G. E. et al., J. Drug Target., 3: 321-340, 1996; van Osdol, W. et al., Cancer Res, 51: 4776-4784, 1991; Sung, C. et al., Cancer Res, 54: 2166-2175, 1994). Together, these observations raise the question whether active targeting is feasible with PEG-conjugated antibodies, or if the previously described increase in tumor localization is a result of non-specific accumulation in the interstitial space surrounding tumor vasculature.

In the development of the present invention, the effect of PEGylation on tumor targeting and immunogenicity of huA33 in a mouse xenograft model was analyzed (Barendswaard, E. C. et al., Int. J. Oncol, 12: 45-53, 1998).

One embodiment of the invention is directed to a method of reducing the effects of colon cancer in a subject. The method comprises administering to a subject a pharmaceutically effective amount of an anti-cancer agent conjugated to a PEG(polyethylene glycol)-conjugated antibody where the antibody is specific for the colon cancer.

The PEG that is conjugated to the antibody may be a substituted or unsubstituted polymer having an average molecular weight of from about 3,000 Daltons to about 40,000 Daltons.

Preferably, the PEG have an average molecular weight of between about 5000 Daltons to about 30,000 Daltons.

In a preferred embodiment, the PEG:antibody ratio of the conjugate is about 35 to 1 or less. For example, the PEG:antibody ratio may be about 30:1. That is, on the average, each molecule of antibody is conjugated to about 35 PEG molecules or less. Preferably, on average, each antibody molecule is conjugated to about 30 PEG molecules. More preferably, on average, each antibody molecule is conjugated to about 15 PEG molecules.

In a preferred embodiment, each antibody molecule is conjugated to PEG molecules of molecular weight of between about 4,000 Daltons to about 7,000 Daltons and on average, each antibody molecule is conjugated to about 35 PEG molecules or less. Preferably, each antibody molecule is conjugated to about 30 PEG molecules. More preferably, the PEG molecules in the conjugate is about 5000 Dalton average weight.

In another preferred embodiment, each antibody molecule is conjugated to PEG molecules having a molecular weight of between about 7000 Daltons to about 25,000 Daltons and on average, each antibody molecule is conjugated to about 20 PEG molecules or less. Preferably, each antibody molecule is conjugated to about 15 PEG molecules. More preferably, the average weight of PEG molecules in the conjugate is about 12000 or about 20,000 Dalton.

The antibody of the invention may be a monoclonal antibody. The monoclonal antibody maybe, for example, A33, 100-210 or 100-310, all of which are known. See, e.g., U.S. Pat. Nos. 5,712,369; 5,342,757; and 5,565,356. Furthermore, the antibody may be a humanized antibody, a chimeric antibody, a trimeric antibody, a heteromeric antibody or a single chain antibody. It is understood that the term antibody in this application also refers to antibody fragments such as Fab′ fragments and F(ab)₂ fragments.

The antibodies of this invention may be administered in conjunction with an anti-cancer agent. The anti-cancer agent useful in this invention may be any known anti-cancer agent. For example, the anti-cancer agent may be calicheamicin, QFA, BCNU, streptozoicin, vincristine, irinotecan, oxaliplatin or 5-fluorouracil. In a preferred embodiment, the anti-cancer agent is a peptide that specifically inhibits the DNA activity of the colon cancer. The anticancer agent may also be a radioactive isotope. Any radioactive isotope effective as an anti-cancer agent may be used. Preferred isotopes include ¹²⁵I, ¹³¹I, ⁹⁹Tc, ⁹⁰Y and ¹¹¹In.

Another embodiment of the invention is directed to an anti-cancer agent-antibody conjugate. The antibody of the conjugate is a PEG(polyethylene glycol)-conjugated antibody (i.e., PEGylated antibody). The anticancer agent-antibody conjugate is integrated into colon cancer cells when it is put into contact with the colon cancer cells. The PEGylated antibody may be an antibody conjugated to one or more PEG molecule wherein said PEG molecule is a substituted or unsubstituted polymer having a molecular weight of from about 3,000 Daltons to about 40,000 Daltons. Preferably, the PEG molecule is a substituted or unsubstituted polymer having a molecular weight of from about 5,000 Daltons to about 30,000 Daltons.

### BRIEF DESCRIPTION OF THE DRAWINGS

FIG. 1 depicts in Panel A: SDS-PAGE analysis of huA33 conjugates with PEG 5 or PEG 20 in different molar PEG:antibody reactant ratios. Conjugates with PEG 12 in molar PEG:antibody ratios from 5 to 100 are depicted in panel B.

FIG. 2 depicts a plot of binding activity vs. PEG:antibody molar ratio. HuA33 modified with PEG 5 is represented with (∇---) whereas huA33 modified with PEG 20 is represented with (▴---).

FIG. 3 depicts immunogenicity of huA33 and PEG-huA33: Groups of five mice each were injected with 5 μg of either native antibody (□), PEG 5 conjugated huA33 (▾), or PEG 20 huA33 (▴) at the labelled time points. Anti-huA33 binding activity was determined by ELISA using unmodified huA33 as the target antigen. Error bars indicate standard deviation.

FIG. 4 Panels A depicts the elimination of native and PEGylated huA33 antibody from the blood of non-tumor bearing Swiss mice. Panels B depicts the elimination of native and PEGylated 3S193 antibody from the blood of non-tumor bearing Swiss mice. Panel C depicts the distribution of labeled and injected huA33-dose in nude mice bearing SW1222 tumor xenografts of defined size. Panel D depicts the tumor:blood ratios at indicated timepoints after injection of radiolabelled antibody into SW 1222-tumor bearing mice. The data is plotted using the following symbols: huA33 (□);PEG 5 conjugated huA33 (▾); PEG 20 conjugated huA33 (▴); native hu3S193 (◯); PEG 20 conjugated hu3S193(●).

FIG. 5 depicts organ distribution of different huA33 preparations as percent injected dose per gram of tissue. Tumor-bearing nude mice were injected with 5 μg of ¹³¹I-labelled native huA33 (white), PEG 5 conjugated huA33 (black), or PEG 20 conjugated huA33 (hatched).

FIG. 6 shows blocking of radiolabeled PEG 5-huA33 binding to tumor tissue by pre-saturation with unlabelled antibodies: Tumor-bearing nude mice were treated with unmodified huA33 (black) or control antibody hu3S193 (hatched) before being injected ¹³¹I labeled PEG-huA33. Blood, organ and tumor doses were measured at the timepoints indicated and expressed as percent injected dose per gram of tissue. White: control without pre-saturation.

FIG. 7 depicts the results of tissue section analysis of tumors following antibody injection. The rows and column designations are as follows. Row A: native huA33; row B: 3S193; row C: PEG 5-huA33; row D: PEG 12-huA33; row E: PEG 20-huA33; column 1: 1 hour; column 2: 4 hours; column 3: 24 hours; and column 4: 72 hours.

### DETAILED DESCRIPTION OF THE INVENTION

Murine monoclonal antibody A33, (referred to herein as mAb A33) is a monoclonal antibody of the IgG2a isotype which defines a cell-surface antigen referred to as antigen A33 (also referred to as the “A33 antigen”), which is present on greater than 95% of colorectal carcinomas and in normal intestinal mucosa (see Table 1). Among colorectal carcinomas, A33 antigen is expressed with a homogeneous cell surface distribution in greater than 95% of antigen-positive lesions. Moreover, A33 antigen is found in colon cancers regardless of their degree of histologic differentiation and in primary as well as metastatic lesions, including liver metastases.

Antigen A33 has not been detected in the sera of patients with antigen A33-positive colon cancers, in the tumoral secretions of mucinous colon cancers, nor in the supernatant of antigen A33-positive colon cancer cell lines. This suggests that antigen A33 is not a secreted molecule. In the normal gastrointestinal tract, antigen A33 is found in normal small and large intestinal mucosa, showing a uniform cell surface distribution throughout the crypts.

A detailed survey of antigen A33 expression in greater than 300 tumors of diverse histologic types and in normal human adult and fetal tissues has shown the restricted distribution pattern of this antigen. As discussed below, the inventors have found that among neoplastic tissues, antigen A33 is present in colon cancers and in a subset of gastric cancers showing intestinal metaplasia, but not in any of the other epithelial cancers tested. Antigen A33 was not found to be present in sarcomas, lymphoid neoplasms, neuroectodermal tumors and some neuroectodermal tumors. These tissues were consistently found to be antigen A33-negative.

As disclosed in U.S. Pat. No. 5,160,723, the entirety of which is incorporated herein by reference, monoclonal antibody A33 is specific for antigen A33 found on colon cancer cell surfaces, and is therefore specific for colon cancer. As a result, monoclonal antibody A33, and other antibodies which are antigen A33-specific, can be used for colon cancer diagnosis. The antibodies can be conjugated with anti-cancer agents, peptides or radioisotopes and utilized for colon tumor treatment.

TABLE 1
Immunohistochemical Analysis of A33
Antigen Expression in Human Tumors

| | A33-positive | |
|---|---|---|
| Tumor Type | No. Cases | % positive cells |
| Carcinomas | | |
| Colorectal carcinoma | 45/47 | 75 − 100 n = 40 |
| primary tumors | | 25 − 50 n = 5 |
| metastases | 23/25 | 75 − 100 n = 22 |
| | | 25 − 50 n = 1 |
| Gastric carcinoma | 14/30 | |
| signet ring type | 12/12 | 75 − 100 n = 12 |
| Esophageal carcinoma | 2/8 | |
| intestinal type | 1/1 | |
| mucinous type | 1/1 | |
| Pancreatic carcinoma | 0/8 | |
| Lung carcinoma | 0/16 | |
| Breast carcinoma | 0/19 | |
| Renal carcinoma | 0/16 | |
| Bladder carcinoma | 0/19 | |
| Prostate carcinoma | 0/4 | |
| Testicular carcinoma | 0/4 | |
| Ovarian carcinoma | 1/56 | |
| Endometrial carcinoma | 1/4 | |
| Thyroid carcinoma | 0/4 | |
| Liver carcinoma | 0/2 | |
| Larynx carcinoma | 0/2 | |
| Mesothelioma | 1/7 | |
| Neuroendocrine carcinomas | 0/8 | |
| Neuroectodermal tumors | | |
| Melanoma | 1/10 | |
| Gliomas | 0/8 | |
| Neuroblastomas, Glgnb | 0/10 | |
| Sarcomas | | |
| Leiomyosarcoma | 0/7 | |
| MFII | 0/5 | |
| Fibrosarcoma | 0/3 | |
| Liposarcoma | 0/6 | |
| MPNT | 0/6 | |
| Chondrosarcoma | 0/14 | |
| Others | 0/9 | |
| Lymphomas | 0/12 | |

### EXAMPLE 1

PEG Modification and Labeling of Humanized Antibodies.

The A33 antigenic system has shown promising tumor-targeting in clinical trials (Welt, S. et al., J Clin Oncol, 12:1561-1571,1994; Welt, S. et al., Proc Annu Meet Am Soc Clin Oncol. A4891992; Welt, S. et al., J. Clin. Oncol., 14:1787-1797, 1996). To reduce its immunogenicity, the A33 antibody has been fully humanized by CDR-grafting (King, D. J. et al., Br. J. Cancer, 72: 1364-1372, 1995).

The following procedure was used for PEG modification of the humanized antibodies. It is understood that for the purposes of this application, PEG-conjugated antibody, PEG-modified antibody, and PEGylated antibody are equal in meaning. The amount of methoxy-PEG-succinimidyl-succinate of Mᵣ 5,000 Daltons (PEG-5), Mᵣ 12,000 Daltons (PEG-10), or Mᵣ 20,000 Daltons (PEG-20) (Shearwater Polymers, Huntsville, Ala.) needed was measured by weight in the reaction tubes. Ten milligrams of the antibodies to be PEG-conjugated were dissolved in 10 ml of 100 mM sodium phosphate buffer pH 7.4. The antibody used was either humanized A33 (huA33) (King, D. J. et al., Br. J. Cancer, 72:1364-1372, 1995) or humanized 3S193 antibody (negative control, from Kitamura, K. et al., Proc. Natl. Acad. Sci. U.S.A., 91:12957-12961, 1994). After the antibodies were dissolved, the antibody solution was added directly into the reaction tubes.

After the addition of the antibodies, the reagents were immediately mixed vigorously for 30 seconds and then left for 60 minutes at room temperature under moderate shaking. Unreacted PEG was removed by ultrafiltration through a membrane with a molecular weight cut-off of 50,000 Daltons (Biomax cartridges, Millipore, Marlborough, Mass.). For uniformity, the protein concentration of the PEGylated antibody solution was adjusted to 1.0 mg/ml. The purity of the PEGylated antibody was assessed by sodium dodecyl sulfate-polyacrylamide gel electrophoresis (SDS-PAGE) on 6% acrylamide or 4-12% acrylamide gels. After electrophoresis, the proteins were visualized by Coomassie staining (Novex, San Diego, Calif.).

Unreacted primary amines were detected by mixing 150 μl of the purified conjugate (PEG conjugated antibody normalized to 1 mg/ml) with 50 μl of 1 mg/ml fluorescamine (Sigma, St. Louis, Mo.) in acetone. Fluorescence was measured using an excitation wavelength of 360 nm (+/−20 nm) and an emittance wavelength of 460 nm (+/−20 nm) (modified after Ref. Stocks, S. J. et al., Anal.Biochem., 154:232-234, 1986). The proportion of modified primary amines was calculated based on unreacted antibody as a standard.

The binding ability of immunoglobulins (PEG-conjugated and unconjugated controls) to SW1222 tumor cells was detected using erythrocyte-bound protein A (indicator erythrocytes) using standard methods (Pfreundschuh, M. et al., Proc. Natl. Acad. Sci. U.S.A., 75: 5122-5126, 1978; Carey, T. E. et al., Proc. Natl. Acad Sci USA, 73: 3278-3282, 1976). Antibody binding titer was defined as the highest dilution that produced unequivocal rosetting of erythrocytes on tumor cells.

Native and PEG-conjugated antibodies (huA33 and hu3S193) were iodinated (¹³¹I) using the chloramine T method as previously described (Barendswaard, E. C. et al., Int. J. Oncol, 12: 45-53, 1998). Labeled protein was separated from free iodine by chromatography on a Sephadex G25 column saturated with 0.5% bovine serum albumin. The iodination degree, as measured by trichloroacetic acid-precipitable protein-bound ¹³¹I, ranged between about 97% to about 99%. Immunoreactivity of the radio-iodinated antibody preparations was determined as described (Barendswaard, E. C. et al., Int. J. Oncol, 12: 45-53, 1998) and was on average about 34% of the reactivity of unlabelled antibody.

### EXAMPLE 2

Characterization of PEG Modified Antibodies.

Eight week old female outbred CD-1 mice (Charles River Laboratories, Wilmington, Mass.) and eight-week old female athymic NCr-nuBR mice (referred herein as “nude” mice; Taconic, Germantown, N.Y.) were used to characterize PEG modified antibodies.

To determine antibody immunogenicity in mice, groups of five CD-1 mice received four weekly intravenous injections of 25 μg native or PEG-modified huA33 antibody (days 1, 8, 15, and 22). Blood samples were obtained before the first injection and about 5 weeks, about 9 weeks, or about 13 weeks post injection.

Immune reactivity against A33 was assessed by ELISA against huA33. Microtiter plates coated with 10 μg/well of huA33 and blocked with 1% w/v bovine serum albumin were incubated with mouse blood samples in duplicate dilution series. Bound murine antiserum was detected photometrically after incubation with alkaline phosphatase-conjugated rabbit anti-mouse IgG, A, M serum (Sigma, St. Louis, Mo.). Immune reactivity was determined in the subsequent chromogenic reaction as the highest dilution that produced an absorbance greater than twice the background.

To determine tissue dosimetry of [¹³¹I]huA33 in mice, nude mice with xenograft tumors and naive controls were injected intravenously with a single dose of 5 μg (10 μCi) radiolabelled native or PEGylated antibody in 100 μl sterile buffer (0.15 M NaCl, 0.1 M sodium phosphate, pH 7.2). For circulation clearance studies, blood samples were taken repeatedly at the different time points from the retro-orbital plexus opposite of the injection site. For organ distribution and histologic tumor uptake studies, lung, liver, spleen, kidneys, and tumor from sacrificed mice were resected and a blood sample obtained. The radioactive dose was measured by an automated gamma counter (Wallac, Gaithersburg, Md.) and compared to an aliquot of the injected preparation as a standard. In vivo doses were calculated as percent of the injected dose per gram of tissue or blood, using the formula

% injected dose/per g=100 ×[(cpm of sample/sample mass (g)×100)/]cpm of injected dose

Morphological studies of the effects of the PEGylated antibodies was performed using the human colon carcinoma cell line SW 1222 from the cell bank of the Ludwig Institute for Cancer Research at Memorial Sloan Kettering Cancer Center. The SW 1222 cells were cultured under standard conditions as previously described (Barendswaard, E. C. et al., Int. J. Oncol, 12: 45-53, 1998), incorporated by reference.

SW 1222 tumor bearing mice (xenografts mice) were produced by injecting the left thigh muscle of nude mice with 10⁷ washed SW 1222 cells in 150 μl sterile buffer (0.15 M NaCl and 0.1 M sodium phosphate at pH 7.4). The nude mice were kept under normal condition until the tumor mass has attained a size of about 350 mg to about 400 mg.

For the studies in this application, SW 1222-tumor bearing nude mice were treated with native or PEG-conjugated huA33 or 3S193 antibody as indicated, and sacrificed at about 1 hour, about 4 hours, about 24 hours and about 72 hours after injection. Tissues were harvested immediately after euthanasia and embedded in cryomolds filled with OCT compound (Tissue-Tek, Torrance, Calif.). The embedded tissue was snap-frozen in isopentane which had been pre-cooled in dry ice. The frozen blocks were stored at −75° C. until used.

Cryostat sections of 5 μm thickness were prepared by adhesion to slides (Superfrost, Fisher Scientific, Pittsburgh, Pa.). The adhered tissue sections were dried at room temperature for 30 minutes. The dried tissue was fixed with cold acetone (4° C.) for 10 minutes immediately before immunostaining.

Immunohistochemistry was performed with an avidin-biotin system (ABC, Vector Laboratories, Burlington, Calif.). The primary reagent, a goat anti-human antibody (1:100; Jackson Laboratories, West Grove, Pa.), was applied at 4° C. overnight. This application was followed by an application of biotinylated horse anti-goat secondary antibody (1:200, Jackson Laboratories). A negative control slide, made by omitting the goat anti-human antibody, was included for all assays.

The chromogen 3,3′diaminobenzidine (DAB, BioGenex, San Ramon, Calif.) was used for visualization. Endogenous peroxidase was suppressed with 1% H₂O₂ for 30 minutes prior the application of the avidin-biotin complex. The slides were counterstained with Meyer's hematoxylin (Sigma, St. Louis, Mo.) and dehydrated. As a staining control, one slide derived from a control animal treated with buffer solution only was stained directly with huA33, followed by the detection system described. Immunohistochemical staining was graded in 25% increments as follows: −, no staining of tumor cells; +,<25% of tumor cells stained; ++, 25-50%, +++, 50-75%; and ++++, >75% of tumor cells stained.

To optimize the conjugation process, the highest PEG: antibody ratio for each PEG-size that would not diminish antibody binding by more than 50% (one titration step) was used. A decrease of antibody binding by 50% can be compensated for by increasing the amount of antibody used. PEG 5 (Mᵣ 5,000), PEG 12 (Mᵣ 12,000) and PEG 20 (Mᵣ 20,000) were examined in PEG:antibody reactant ratios from 5 to 100. The reaction products were heterogeneous in size, reflecting different conjugation ratios achieved in the reaction. As PEG migrates slower than proteins of the same mass in polyacrylamide gel (Francis, G. E. et al., J. Drug Target., 3: 321-340, 1996), electrophoresis may not reveal the actual molecular weight. Thus, the electrophoresis migration was used only to estimate the amount of unreacted antibody and to compare different PEG preparations.

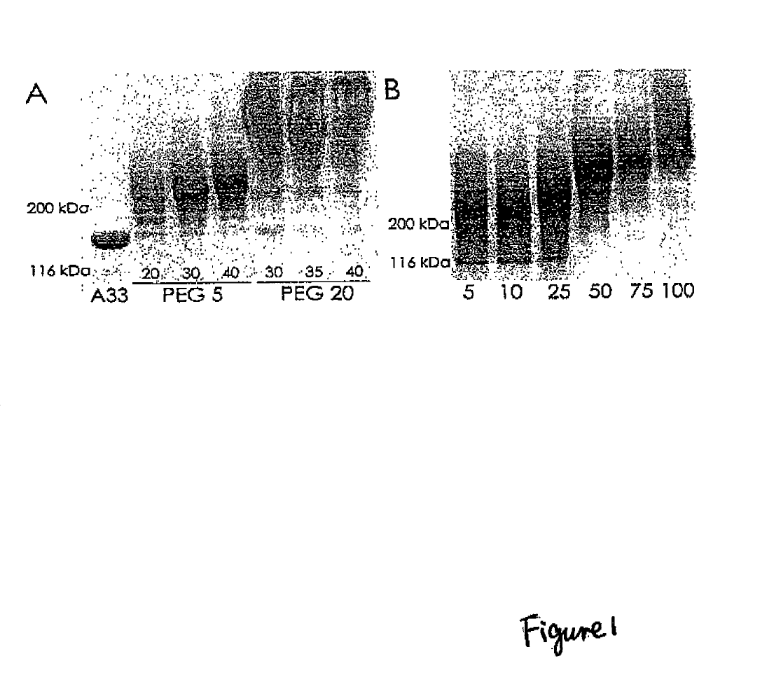

The results of an SDS-PAGE analysis are shown in FIG. 1. Humanized A33 antibody (huA33) conjugated according to the procedure described above was analyzed by SDS-PAGE on6% acrylamide (FIG. 1A) or 4-12% acrylamide gradient (FIG. 1B) tris-glycine gels under non-reducing conditions. In FIG. 1A, huA33 conjugates with PEG 5 (in molar PEG:antibody reactant ratios of20,30, and 40) or PEG20 (in molar PEG:antibody reactant ratios of30, 35, and 40) were analyzed. The first lane of FIG. 1A contains native (unconjugated) huA33 control. Conjugates with PEG 12 in molar PEG:antibody ratios from 5 to 100 were analyzed in 4-12% acrylamide gradient gels in FIG. 1B. The results show that the speed of migration was dependent on the PEG size (FIG. 1A) as well as the molar reactant ratio (FIG. 1B). That is, the speed of migration decreased as PEG size or molar reactant ratio is increased. The actual percentage of modified primary amines was determined by the fluorescamine assay of Stocks et al. (Stocks, S. J. et al., Anal.Biochem., 154: 232-234, 1986). The fluorescamine assay demonstrated almost complete reaction (conjugation) of PEG when the PEG:antibody ratios were about 70:1 or less. This effective conjugation ratio is close to the actual PEG:antibody ratios used in the studies of this application.

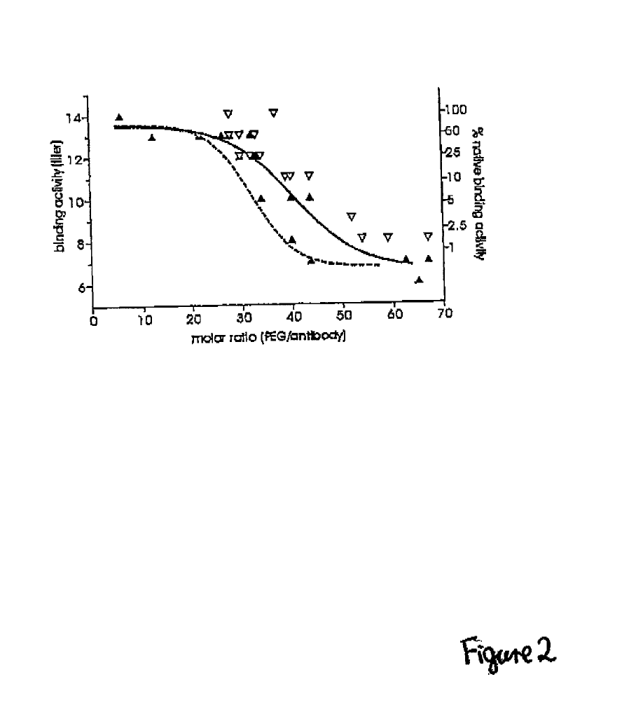

To determine the optimal level of PEG-conjugation, the immunoreactivity of PEG/antibody conjugates was determined by mixed hemadsorption assay on A33-antigen positive SW1222 colon cancer cells. The results were analyzed by non-linear regression tests and expressed as PEG/antibody ratios and binding activities in FIG. 2. Average PEG: antibody ratios of the conjugate were measured by fluorescamine assay while the antibody binding titer was determined by mixed hemadsorption assay. In FIG. 2, HuA33 modified with PEG 5 is represented with (∇---) whereas huA33 modified with PEG 20 is represented with (▴---). The left hand scale of FIG. 2 represents dilution titer while the right hand scale of FIG. 2 represents percent of activity (unmodified huA33=100%). From these results, molar ratios of PEG:antibodies were selected for subsequent studies.

PEG:antibody ratios of up to 30:1 (32-34% of primary amines modified) for PEG 5 and up to 15:1 (18% of primary amines modified) for PEG 12 and PEG 20, showed no inhibition of antibody binding that exceeded the limit of one titration step (FIG. 2). Thus, these ratios were selected for subsequent experiments. At 4° C. the conjugates were stable for at least about eight weeks according to gel electrophoresis and activity tests.

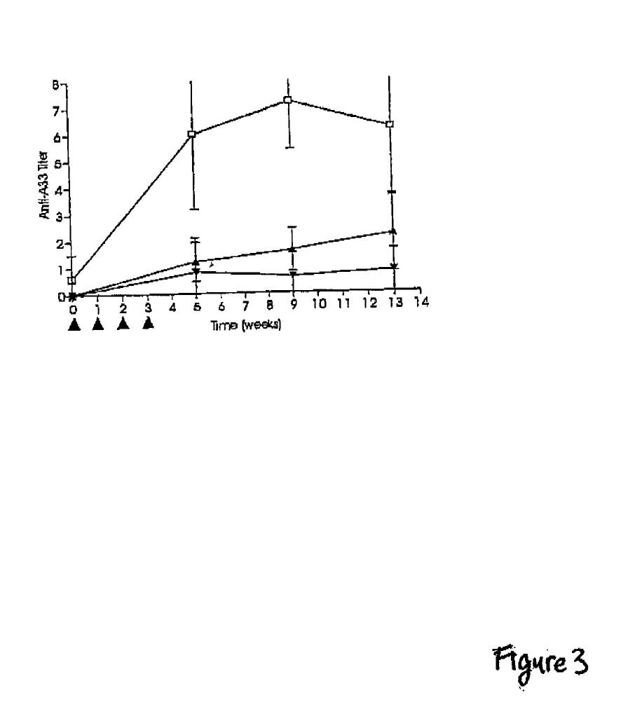

To determine the effects of PEGylation, the immunogenicity of the antibodies was tested on CD-1 mice. Briefly, four groups of five mice each were injected with 5 ug (250 μg/kg) of antibody at week 0, week 1, week 2 and week 3. Group 1 mice received native (□) hu33A antibody injections. Group 2 mice received PEG 5 conjugated antibody (▾) injections. Group 3 mice received PEG 20 conjugated (A) huA33 antibody preparations. The results are plotted in FIG. 3. The anti-human antibody titers were determined at 0 week (before injection), 5 weeks, 9 weeks, and 13 weeks by ELISA assays. Anti-huA33 binding activity was determined in an ELISA using unmodified huA33 as the target antigen. The results of this test are depicted in FIG. 3. The error bars in FIG. 3 indicate standard deviation. The results of one-tailed paired t-test are as follows:

native vs. PEG 5-huA33, P=0.0217

native vs. PEG 20-huA33, P=0.0211

PEG 5- vs. PEG 20-huA33, P>0.05

As can be seen in FIG. 3, mice that received native huA33 produced increased levels of anti-huA33 antibodies on week 5 (day 35), and 4 of 5 mice reached a maximum titer of 256 on week 9 (day 63). With both PEG conjugated antibody preparations, however, a titer of 4 was not exceeded, and the highest titer was only observed on week 13 (day 91) (significance level of difference to native huA33: PEG 20-huA33, P<0.05; PEG 5-huA33, P<0.01; no significant difference between PEG-huA33 preparations). Thus, PEG modification reduces immunogenicity of huA33.

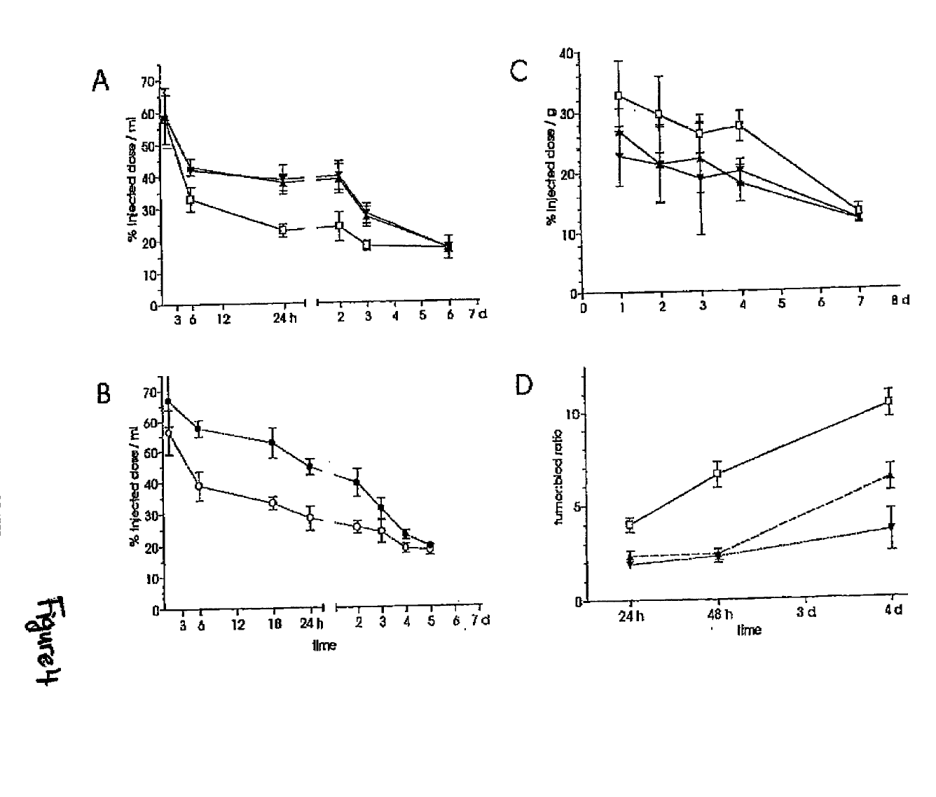

To determine the effects of PEGylation on the levels of antibodies in a subject, radioactive antibodies were injected into mice and the levels of radioactivity was monitored. The results are shown in FIG. 4. Briefly, mice in groups of five were injected with 5 μg of one of the following ¹³¹I-labelled antibody preparations: (1) native huA33 (□); (2) PEG 5 conjugated huA33 (▾); (3) PEG 20 conjugated huA33 (▴); (4) native hu3S193(◯); or (5) PEG 20 conjugated hu3S193 (●). Blood and/or tumor tissue was obtained at the time points indicated in FIG. 4. The radioactive dose per gram was measured and normalized for the injected dose. Panels A and B in FIG. 4 measured the elimination of native and PEGylation antibody preparation from the blood of non-tumor bearing Swiss mice. Panel A and B show the results of experiments performed using HuA33 antibody or the 3S193 antibodies respectively. The PEG:antibody ratios were 15:1 for PEG 20-huA33, 30:1 for PEG 5-huA33, and 40:1 for PEG 20-3S193. Panel A and B each represents summarized data from three experiments.

At the selected PEGylation ratios of 15:1 for PEG 20 and 30:1 for PEG 5, only a slight increase in serum half life was observed compared to non-PEGylated A33 antibody, resulting in dose levels of PEGylated antibody that were about 125% of the corresponding dose of native huA33 after 6 h and about 165% after 48 hours, converging thereafter (paired one-tailed t-test: PEG 5, P=0.0021; PEG 20, P=0.0018). Only when a considerably higher conjugation ratio, 40:1 with PEG 20, was used on the isotype control antibody 3S193 was a more marked increase in serum half-life observed (FIG. 4B).

To determine tumor and organ uptake, groups of five SW1222 xenograft-bearing nude mice each were injected with 0.5 μg of trace-labelled native or PEGylated huA3 3. Animals from each group were sacrificed at 24, 48, 72, 96 and 168 hours post-injection to measure blood, tumor and organ doses, which were expressed as percent injected dose per milliliter of blood or gram of tissue, respectively. With all antibody preparations, the maximum tumor dose was reached at 24 hours, declining thereafter (FIG. 4C, summarized data from 2 experiments). Tumor uptake of PEGylated huA33 reached 73% to 82% of the uptake of native huA33 at corresponding time points (paired one-tailed t-test for difference between native huA33 and either PEG-preparation, P<0.01). Tumor:blood ratios, shown in FIG. 4D, also were significantly higher with native huA33 compared to either PEG-conjugate (P=0.0413 for PEG 5 and P=0.0285 for PEG 20), while no significant difference was found between different PEG-conjugates. The results of the experiments shown in FIG. 4 indicate that PEG-conjugation moderately increases the circulating dose of humanized antibodies, but reduces huA33 dose in tumor.

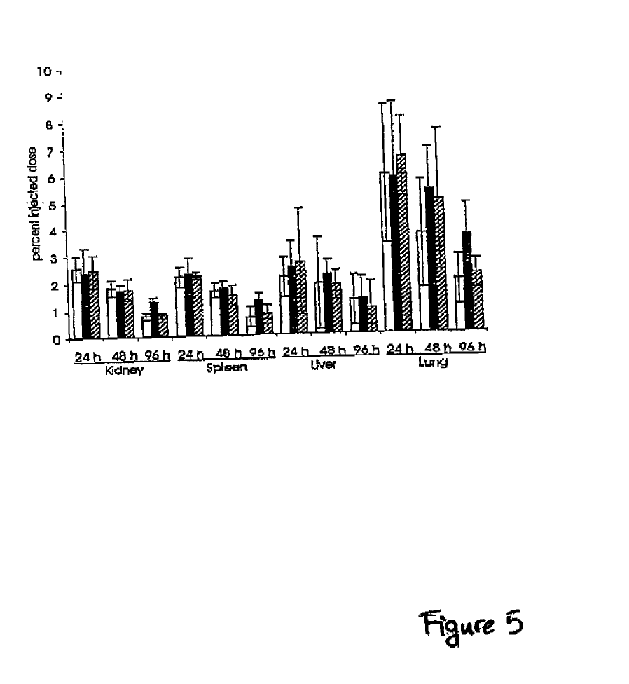

In organ tissues, small differences in uptake were observed between native and PEGylated huA33. The results are depicted in FIG. 5. Briefly, tumor-bearing nude mice were injected with 5 μg of ¹³¹I-labelled native huA33 (white), PEG 5 conjugated huA33 (black), or PEG 20 conjugated huA33 (hatched) antibody. The data plotted represents percent injected dose per gram of tissue. The difference in the organ uptake was statistically significant at the last time point, 96 hours, which was well into the elimination phase. Notably, uptake of the PEG 5-huA33 conjugate exceeded that of both native or PEG 20-conjugated huA33 in lung (P<0.05), kidney, and spleen (both, P<0.01, FIG. 5).

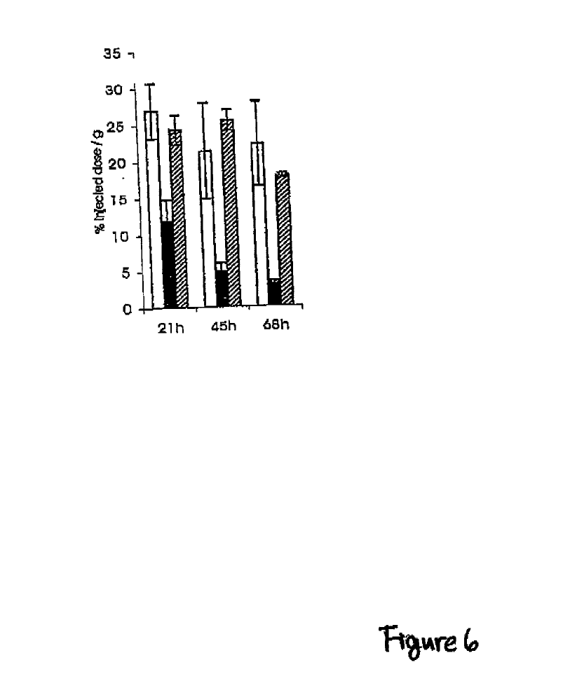

In order to assess the immunologic specificity of antibody localization to tumor in vivo, the previous xenograft experiment was modified by pre-treating mice with excess native (unlabelled, non-PEGylated) antibody to pre-saturate antigenic sites. To perform this presaturation, tumor-bearing nude mice were injected with 250 μg of either huA33 or hu3193 control antibody. Six hours later 5 μg of ¹³¹I-labelled PEG 20-huA33 was injected, and animals were sacrificed after 21, 45, and 68 hours. Blood, organ and tumor doses were measured and expressed as percent injected dose per gram of tissue. The result is shown in FIG. 6 wherein mice blocked with unmodified huA33 antibody are represented by black bars, mice blocked with control hu3S193 antibody (hatched) is shown in hatched bars and control mice without pre-saturation are shown in white bars. No significant difference between the two pre-treatment groups was observed for radioactive doses in blood, kidney, spleen, liver, or lung. In tumor tissue, however, blocking with huA33 significantly reduced ¹³¹I-PEG-huA33 binding down to the levels of nonspecific binding in organ tissues (P=0.0024 for difference to unblocked control), whereas pre-treatment with unlabelled hu3S193 control antibody had no effect on PEG-A33 binding (P=0.3889 for difference to control. P=0.0108 for difference to huA38-block, FIG. 6).

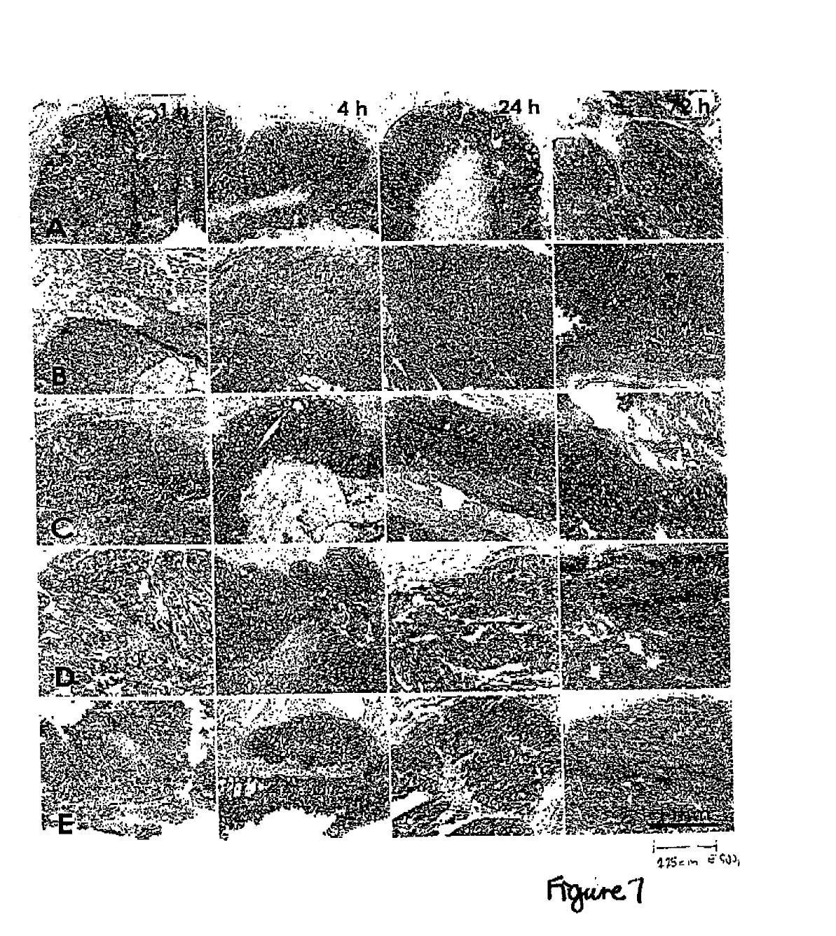

To determine if PEG conjugated antibody shows the same microdistribution in tumors as unconjugated antibodies, tumors were examined for antibody distribution. Briefly, mice bearing tumors of defined size were injected with 5 μg of the antibody solutions. At different time-points, tumors were resected, and thin sections were subsequently stained with IgG specific goat anti-human primary and biotinylated horse anti-goat secondary antibodies and a streptavidin-alkaline phosphatase conjugate, which was detected by reaction with a chromogenic substrate. The results of the immunohistochemical staining are shown in Table 6 and FIG. 7. All tissues showed variable degrees of intravascular and stromal staining due to the presence of humanized antibody in blood vessels and connective tissue. No staining was present in the neoplastic xenograft tumor cells of the animals treated with 3S193. Mice treated with native huA33 showed intense tumor cell staining at all time points. With PEG 5-modified huA33, homogeneous tumor staining was observed after 4 hours, and with PEG 12 and PEG 20-conjugates after 24 hours. The results show that PEG conjugated huA33 antibodies have the same distribution in the tumors as unconjugated antibodies.

TABLE 6
Evaluation of slides from FIG. 7 for distribution and
intensity of tumor staining

| | 1 hr | 4 h | 24 h | 72 h |
|---|---|---|---|---|
| Buffer | − | − | − | − |
| Native hu3S193 | − | − | − | − |
| (FIG. 18, row B) | | | | |
| Native huA33 | ++++ | ++++ | ++++ | ++++ |
| (FIG. 18, row A) | | | | |
| PEG 5-huA33 | + | ++++ | ++++ | +++ |
| (FIG. 18, row C) | | | | |
| PEG 12-huA33 | ++ | ++++ | ++++ | ++++ |
| (FIG. 18, row D) | | | | |
| PEG 20-huA33 | ++/+++ | +++ | ++++ | +++ |
| (FIG. 18, row E) | | | | |

Completely negative (−), <25% (+), 25 to 50% (++), 50 to 75% (+++), and >75% (++++) of tumor cells stained.

This study demonstrates that PEG-conjugated huA33 antibody localizes to tumor tissue in vivo with immunological specificity. At conjugation ratios sufficient to suppress immunogenicity, PEG-huA33 showed homogeneous targeting to tumor tissue comparable to the native antibody. However, the amount of PEG-conjugated antibody present in the tumor was only about 75% of that achieved with the native (nonconjugated) antibody. The elimination rate from tumors showed no difference between PEG-conjugated and nonconjugated antibodies but the tumor:blood ratios of the PEG conjugates were about one-half those of unmodified huA33. This ratio increased over time for all three preparations of PEG-conjugated antibodies as circulating antibody was eliminated from the vascular compartment.

In this study, three phases in the micro-localization of non-PEGylated huA33 were observed: a) initial targeting: as early as one hour post injection huA33 localized with high intensity to peripheral tumor cells; b) distribution in tumor tissue: heterogeneous staining throughout the tumor nodule was observed after about 4 hour, and homogeneous staining was achieved at about 24 hour; and c) clearance of nonspecific staining: stroma and vasculature were almost completely unstained after 72 hour, whole tumor tissue remained homogeneously stained. With PEGylated huA33, the targeting process followed the same consecutive pattern but was delayed by three to 24 hours.

Theoretical models have predicted that antibodies may not be able to achieve tumor-targeting beyond the periphery of a tumor, as the concentration gradient toward its core would be insufficient to overcome the outward directed convective pressure gradient (Jain, R. K. Cancer Res, 50: 814s-819s, 1990; van Osdol, W. et al., Cancer Res, 51: 4776-4784, 1991). However, this is not the case with the A33 antigenic system, since homogeneous distribution of A33 antibody throughout colon cancer tissue has been demonstrated in mice and humans (Welt, S. et al., J. Clin. Oncol, 8: 1894-1906, 1990; Barendswaard, E. C. et al., Int. J. Oncol, 12: 45-53, 1998). The present study confirms these findings for PEG-huA33. A possible explanation for the fast and homogeneous distribution may be the high internalization rate of antigen-antibody complexes documented for A33 (Daghighian, F. et al., J. Nucl. Med., 3 7:1052-1057, 1996). The binding-site barrier model postulates that a high-affinity antibody to an abundantly expressed antigen will form a gradient from periphery to center, with most antibody binding at the entry site in the periphery, preventing further diffusion into tumor tissue (van Osdol, W. et al., Cancer Res, 51: 4776-4784, 1991). Thus, the internalization of antigen-antibody complexes and the consequent depletion of antigenic binding sites permit deeper penetration of antibody into the tumor. As long as a sufficient amount of antibody is present over time, antibody localization would thus progressively advance towards the core of a tumor nodule.

Several authors have described increased passive, i.e., not antigen-specific, tumor targeting as an effect of PEGylation of various proteins and non-protein drugs (Vaage, J. et al., Br. J. Cancer, 75: 482-486,1997; Westerman, P. et al., Int. J. Cancer, 76: 842-850,1998; Senter, P. D. et al., Bioconjug. Chem., 6: 389-394, 1995). To exclude the possibility that the observed tumor localization represented mere passive uptake, antigen-specific binding of PEGylated huA33 in tumor-xenografted mice was demonstrated, by pre-saturation of antigenic sites with unconjugated, unlabelled antibodies. Native huA33 reduced subsequent detection of radiolabelled PEG-huA33 to the level of non-specific uptake in organ tissues, while pre-treatment with a control antibody had no significant effect. In addition, after pre-saturation with the control antibody, tumor:blood and tumor:organ ratios were highest during the elimination phase of the antibody, which is consistent with a binding force that retained PEG-huA33 in tumor against a concentration gradient. These results show that PEG-huA33 targeting is immunologically specific and not due to non-specific pharmacokinetic characteristics of certain PEGylated proteins.

The objective of PEG-conjugation in this study was to reduce the immunogenicity of a therapeutic antibody. In clinical trials, the humanized AS3 antibody elicited a human-anti-human antibody response in a proportion of patients (Welt, S. et al., Proc. Annu Meet Am Soc Clin Oncol, 16: A15631997). The immunogenicity of huA33 as a xenogenic protein in mice was reduced by more than 95% after modification of about 34% of primary amines with PEG 5 or of about 18% of primary amines with PEG 20. However, the latter conjugate displayed slightly higher immunogenicity, with increasing titers over time. While not statistically significant, this difference might be meaningful, and may be due to the lower conjugation degree rather than the higher PEG size. This would confirm previous findings that reduction in immunogenicity depends on the degree of PEGylation. In one study, for example, more than 60% of primary amines needed to be modified to suppress reactivity of ovalbumin with existing ovalbumin-specific antibodies, while tolerance against immunization in mice could be induced at a PEG:protein ratio of less than 30% (Saito, T. et al., J. Biomater. Sci. Polym. Ed., 8: 311-321, 1996).

Using the sulfhydryl-methoxy-PEG method, the conjugation conditions were optimized so as to achieve the highest possible PEGylation degree while leaving little or no antibody unconjugated and not incurring more than 50% (one titer step) loss in antibody binding activity. However, as tumor uptake of A33 antibody is dependent on its antigen specificity, lower binding activity of PEGylated A33 was likely to contribute to the reduced tumor:blood ratio compared to native antibodies. New conjugation techniques such as the linker-less Tresyl-mPEG method (Francis, G. E. et al., J. Drug Target., 3: 321-340, 1996) and newly developed di- or polyvalent PEG types (Inada, Y. et al., Trends Biotechnol., 18: 86-91, 1995) are also contemplated by this invention. Such techniques may offer ways to improve the balance between reduced immunogenicity and preserved function in future investigations.

PEGylation caused a modest increase in the circulating half life of huA33 in comparison to the native antibody. A more marked increase in circulation time was observed only at PEG:antibody conjugation ratios that significantly reduced immunoreactivity. The immediate effects of PEG-conjugation on circulating dose and tumor localization are determined mainly by two factors: protection from enzymatic degradation and reduced diffusion due to increased size (Inada, Y. et al., Trends Biotechnol., 18: 86-91, 1995). Protection from degradation should prolong the circulating half-life of a PEGylated molecule independent of its size. The increase in effective diameter prolongs circulating half-life. This effect will be most prominent with small proteins that pass the renal filter in their native form but are retained after PEGylation. This is the case with antibody fragments, which have shown a marked increase in serum half-life after PEGylation, whereas complete IgG antibodies pass the renal filter neither in their native nor in the PEGylated form (Kitamura, K. et al., Cancer Res, 51: 4310-4315, 1991; Delgado, C. et al., Br. J. Cancer, 73: 175-182, 1996; Eno-Amooquaye, E. A. et al., Br. J. Cancer, 73: 1323-1327, 1996; Pedley, R. B., et al., Br. J. Cancer, 70: 1126-1130, 1994). On the other hand, an increase in diameter also impedes the diffusion of a protein of any size in perivascular space and tumor tissue, reducing its capability of tumor targeting and penetration. On balance, the effect of the increased diameter on tumor localization is more favorable for smaller antibody fragments, which are excreted rapidly in their non-PEGylated forms, while in larger molecules such as complete IgG the impeding effect on tumor targeting prevails.

Other embodiments and uses of the invention will be apparent to those skilled in the art from consideration of the specification and practice of the invention disclosed herein. All U.S. patents and applications and other references noted herein are specifically incorporated by reference. The specification and examples should be considered exemplary only with the true scope and spirit of the invention indicated by the following claims.

We claim:

1. A method of reducing the effects of colon cancer in a subject comprising administering to said subject a pharmaceutically effective amount of an anti-cancer agent conjugated to a PEG(polyethylene glycol)-conjugated antibody, wherein said antibody is specific for said colon cancer.

2. The method of claim 1 wherein said PEG-conjugated antibody is an antibody conjugated to one or more PEG molecule wherein said PEG molecule is a substituted or unsubstituted polymer having a molecular weight of from about 3,000 Daltons to about 40,000 Daltons.

3. The method of claim 1 wherein said PEG-conjugated antibody is an antibody conjugated to one or more PEG molecule wherein said PEG molecule is a substituted or unsubstituted polymer having a molecular weight of from about 5,000 Daltons to about 30,000 Daltons.

4. The method of claim 1 wherein said PEG-conjugated antibody is conjugated to PEG at a PEG:antibody molar ratio of less than about 30:1.

5. The method of claim 1 wherein said PEG-conjugated antibody is conjugated to PEG at a PEG:antibody molar ratio of less than about 15:1.

6. The method of claim 1 wherein said PEG-conjugated antibody is an antibody conjugated to one or more PEG molecule wherein said PEG molecule is a substituted or unsubstituted polymer having a molecular weight of about 4,000 Daltons to about 7,000 Daltons and wherein said antibody is conjugated to PEG at a PEG:antibody molar ratio of less than about 35:1.

7. The method of claim 1 wherein said PEG-conjugated antibody is an antibody conjugated to one or more PEG molecule wherein said PEG molecule is a substituted or unsubstituted polymer having a molecular weight of about 7,000 Daltons to about 25,000 Daltons and wherein said antibody is conjugated to PEG at a PEG: antibody molar ratio of less than about 20:1.

8. The method of claim 1 wherein said antibody is monoclonal antibody.

9. The method of claim 9 wherein said monoclonal antibody is selected from the group consisting of monoclonal antibodies A33, 100-210 and 100-310.

10. The method of claim 1 wherein said antibody is selected from the group consisting of a humanized antibody, a chimeric antibody, a trimeric antibody, a heteromeric antibody, a single chain antibody and an antibody fragment.

11. The method of claim 1 wherein said anti-cancer agent is a drug selected from the group consisting of calicheamicin, QFA, BCNU, streptozoicin, vincristine, irinotecan, oxiplatin, and 5-fluorouracil.

12. The method of claim 1 wherein said anti-cancer agent is a peptide that specifically inhibits DNA activity of said colon cancer.

13. The method of claim 1 wherein said anti-cancer agent is a radioactive isotope.

14. The method of claim 13 wherein said radioactive isotope is selected from the group consisting of ¹²⁵I, ¹³¹I, ⁹⁹Tc, ⁹⁰Y and ¹¹¹In.

15. An anti-cancer agent-antibody conjugate which is integrated into colon cancer cells when put into contact with said cells wherein said antibody is a PEG(polyethylene glycol)-conjugated antibody.

16. The conjugate of claim 15 wherein said PEG-conjugated antibody is an antibody conjugated to one or more PEG molecule wherein said PEG molecule is a substituted or unsubstituted polymer having a molecular weight of from about 3,000 Daltons to about 40,000 Daltons.

17. The conjugate of claim 15 wherein said PEG-conjugated antibody is an antibody conjugated to one or more PEG molecule wherein said PEG molecule is a substituted or unsubstituted polymer having a molecular weight of from about 5,000 Daltons to about 30,000 Daltons.

18. The conjugate of claim 15 wherein said PEG-conjugated antibody is conjugated to PEG at a PEG:antibody molar ratio of less than about 30:1.

19. The conjugate of claim 15 wherein said PEG-conjugated antibody is conjugated to PEG at a PEG:antibody molar ratio of less than about 15:1.

20. The conjugate of claim 15 wherein said PEG-conjugated antibody is an antibody conjugated to one or more PEG molecule wherein said PEG molecule is a substituted or unsubstituted polymer having a molecular weight of about 4,000 Daltons to about 7,000 Daltons and wherein said antibody is conjugated to PEG at a PEG:antibody molar ratio of less than about 35:1.

21. The conjugate of claim 15 wherein said PEG-conjugated antibody is an antibody conjugated to one or more PEG molecule and wherein said PEG molecule is a substituted or unsubstituted polymer having a molecular weight of about 7,000 Daltons to about 25,000 Daltons and wherein said antibody is conjugated to PEG at a PEG:antibody molar ratio of less than about 20:1.

22. The conjugate of claim 15 wherein said antibody is monoclonal antibody.

23. The conjugate of claim 15 wherein said monoclonal antibody is selected from the group consisting of monoclonal antibodies A33, 100-210 and 100-310.

24. The conjugate of claim 15 wherein said antibody is selected from the group consisting of a humanized antibody, a chimeric antibody, a trimeric antibody, a heteromeric antibody, a single chain antibody, and an antibody fragment.

25. The conjugate of claim 15 wherein said anti-cancer agent is a drug selected from the group consisting of calicheamicin, QFA, BCNU, streptozoicin, vincristine and 5-fluorouracil.

26. The conjugate of claim 15 wherein said anti-cancer agent is a peptide that specifically inhibits DNA activity of said colon cancer.

27. The conjugate of claim 15 wherein said anti-cancer agent is a radioactive isotope.

28. The conjugate of claim 27 wherein said radioactive isotope is selected from the group consisting of ¹²⁵I, ¹³¹I, ⁹⁹Tc, 90Y and ¹¹¹In.

29. A method for determining if colon cancer is present, comprising contacting a sample believed to contain colon cancer cells with the conjugate of claim 15, and determining amount of said conjugate taken up by cells in said sample, as a determination of cancer in said sample.

30. A method for diagnosing presence or location of colon cancer in a subject, comprising administering an amount of the conjugate of claim 15 to said subject, and determining uptake or locus of uptake of said conjugate by said patient as a determination of presence or locus of cancer in said subject.
